(19) United States
(12) Patent Application Publication
(10) Pub. No.: US 2005/0089554 A1
(43) Pub. Date: Apr. 28, 2005

(54) Apparatus and method for enhancing transdermal drug delivery

(76) Inventors: Michel J.N. Cormier, Mountain View, CA (US); WeiQi Lin, Palo Alto, CA (US); Juanita Johnson, Belmont, CA (US); Kofi Nyam, Palo Alto, CA (US)

Correspondence Address: Francis Law Group, 1942 Embarcadero, Oakland, CA, 94606, US

(21) Appl. No.: 10/971,430

(22) Filed: Oct. 21, 2004

Related U.S. Application Data
(60) Provisional application No. 60/514,433, filed on Oct. 24, 2003.

(51) Int. Cl. A61F 13/02; A61L 15/16; A61K 9/70; A61M 31/00

(52) U.S. Cl. 424/448; 604/500

(57) ABSTRACT

An apparatus for transdermally delivering a biologically active agent comprising (i) a gel pack containing a hydrogel formulation and (ii) a microprojection member having top and bottom surfaces, a plurality of openings that extend through the microprojection member and a plurality of stratum corneum-piercing microprotrusions that project from said bottom surface of the microprojection member, the microprojection member being adapted to receive the gel pack whereby the hydrogel formulation flows through the microprojection member openings. Preferably, the hydrogel formulation comprises a water-based hydrogel.

### CROSS-REFERENCE TO RELATED APPLICATIONS

This application claims the benefit of U.S. Provisional Application No. 60/514,433, filed Oct. 24, 2003.

### FIELD OF THE PRESENT INVENTION

The present invention relates generally to transdermal drug delivery systems and methods. More particularly, the invention relates to a percutaneous drug delivery apparatus having extended drug delivery and a method for using same.

### BACKGROUND OF THE INVENTION

Drugs are most conventionally administered either orally or by injection. Unfortunately, many drugs are completely ineffective or have radically reduced efficacy when orally administered since they either are not absorbed or are adversely affected before entering the bloodstream and thus do not possess the desired activity. On the other hand, the direct injection of the drug into the bloodstream, while assuring no modification of the drug during administration, is a difficult, inconvenient, painful and uncomfortable procedure which sometimes results in poor patient compliance.

Hence, in principle, transdermal delivery provides for a method of administering drugs that would otherwise need to be delivered via hypodermic injection or intravenous infusion. Transdermal drug delivery offers improvements in both of these areas. Transdermal delivery when compared to oral delivery avoids the harsh environment of the digestive tract, bypasses gastrointestinal drug metabolism, reduces first-pass effects, and avoids the possible deactivation by digestive and liver enzymes. Conversely, the digestive tract is not subjected to the drug during transdermal administration. Indeed, many drugs such as aspirin have an adverse effect on the digestive tract. However, in many instances, the rate of delivery or flux of many agents via the passive transdermal route is too limited to be therapeutically effective.

The word “transdermal” is used herein as a generic term referring to passage of an agent across the skin layers. The word “transdermal” refers to delivery of an agent (e.g., a therapeutic agent such as a drug or an immunologically active agent such as a vaccine) through the skin to the local tissue or systemic circulatory system without substantial cutting or penetration of the skin, such as cutting with a surgical knife or piercing the skin with a hypodermic needle. Transdermal agent delivery includes delivery via passive diffusion as well as delivery based upon external energy sources including electricity (e.g., iontophoresis) and ultrasound (e.g., phonophoresis). While drugs do diffuse across both the stratum corneum and the epidermis, the rate of diffusion through the stratum corneum is often the limiting step. Many compounds, in order to achieve an effective dose, require higher delivery rates than can be achieved by simple passive transdermal diffusion. When compared to injections, transdermal agent delivery eliminates the associated pain and reduces the possibility of infection.

Theoretically, the transdermal route of agent administration could be advantageous for the delivery of many therapeutic proteins, since proteins are susceptible to gastrointestinal degradation and exhibit poor gastrointestinal uptake and transdermal devices are more acceptable to patients than injections. However, the transdermal flux of medically useful peptides and proteins is often insufficient to be therapeutically effective due to the relatively large size/molecular weight of these molecules. Often the delivery rate or flux is insufficient to produce the desired effect or the agent is degraded prior to reaching the target site, for example while in the patient's bloodstream.

Transdermal drug delivery systems generally rely on passive diffusion to administer the drug while active transdermal drug delivery systems rely on an external energy source (e.g., electricity) to deliver the drug. Passive transdermal drug delivery systems are more common. Passive transdermal systems have a drug reservoir containing a high concentration of drug. The reservoir is adapted to contact the skin, which enables the drug to diffuse through the skin and into the body tissues or bloodstream of a patient. The transdermal drug flux is dependent upon the condition of the skin, the size and physical/chemical properties of the drug molecule, and the concentration gradient across the skin. Because of the low permeability of the skin to many drugs, transdermal delivery has had limited applications. This low permeability is attributed primarily to the stratum corneum, the outermost skin layer which consists of flat, dead cells filled with keratin fibers (keratinocytes) surrounded by lipid bilayers. This highly-ordered structure of the lipid bilayers confers a relatively impermeable character to the stratum corneum.

One common method of increasing the passive transdermal diffusional drug flux involves pre-treating the skin with, or co-delivering with the drug, a skin permeation enhancer. A permeation enhancer, when applied to a body surface through which the drug is delivered, enhances the flux of the drug therethrough. However, the efficacy of these methods in enhancing transdermal protein flux has been limited, at least for the larger proteins, due to their size.

Active transport systems use an external energy source to assist drug flux through the stratum corneum. One such enhancement for transdermal drug delivery is referred to as “electrotransport.” This mechanism uses an electrical potential, which results in the application of electric current to aid in the transport of the agent through a body surface, such as skin. Other active transport systems use ultrasound (i.e., phonophoresis) and heat as the external energy source.

There also have been many techniques and systems developed to mechanically penetrate or disrupt the outermost skin layers thereby creating pathways into the skin in order to enhance the amount of agent being transdermally delivered. Early vaccination devices known as scarifiers generally include a plurality of tines or needles that were applied to the skin to and scratch or make small cuts in the area of application. The vaccine was applied either topically on the skin, such as disclosed in U.S. Pat. No. 5,487,726, or as a wetted liquid applied to the scarifier tines, such as, disclosed in U.S. Pat. Nos. 4,453,926, 4,109,655, and 3,136,314.

Scarifiers have been suggested for intradermal vaccine delivery, in part, because only very small amounts of the vaccine need to be delivered into the skin to be effective in immunizing the patient. Further, the amount of vaccine delivered is not particularly critical since an excess amount also achieves satisfactory immunization.

However, a serious disadvantage in using a scarifier to deliver a drug is the difficulty in determining the transdermal drug flux and the resulting dosage delivered. Also, due to the elastic, deforming and resilient nature of skin to deflect and resist puncturing, the tiny piercing elements often do not uniformly penetrate the skin and/or are wiped free of a liquid coating of an agent upon skin penetration.

Additionally, due to the self-healing process of the skin, the punctures or slits made in the skin tend to close up after removal of the piercing elements from the stratum corneum. Thus, the elastic nature of the skin acts to remove the active agent liquid coating that has been applied to the tiny piercing elements upon penetration of these elements into the skin. Furthermore, the tiny slits formed by the piercing elements heal quickly after removal of the device, thus limiting the passage of the liquid agent solution through the passageways created by the piercing elements and in turn limiting the transdermal flux of such devices.

Other systems and apparatus that employ tiny skin piercing elements to enhance transdermal drug delivery are disclosed in European Patent EP 0 407063A1, U.S. Pat. Nos. 5,879,326, 3,814,097, 5,279,54, 5,250,023, 3,964,482, Reissue No. 25,637, and PCT Publication Nos. WO 96/37155, WO 96/37256, WO 96/17648, WO 97/03718, WO 98/11937, WO 98/00193, WO 97/48440, WO 97/48441, WO 97/48442, WO 98/00193, WO 99/64580, WO 98/28037, WO 98/29298, and WO 98/29365; all incorporated herein by reference in their entirety.

The disclosed systems and apparatus employ piercing elements of various shapes and sizes to pierce the outermost layer (i.e., the stratum corneum) of the skin. The piercing elements disclosed in these references generally extend perpendicularly from a thin, flat member, such as a pad or sheet. The piercing elements in some of these devices are extremely small, some having a microprojection length of only about 25-400 microns and a microprojection thickness of only about 5-50 microns. These tiny piercing/cutting elements make correspondingly small microslits/microcuts in the stratum corneum for enhancing transdermal agent delivery therethrough.

The disclosed systems further typically include a reservoir for holding the drug and also a delivery system to transfer the drug from the reservoir through the stratum corneum, such as by hollow tines of the device itself. One example of such a device is disclosed in WO 93/17754, which has a liquid drug reservoir. The reservoir must however be pressurized to force the liquid drug through the tiny tubular elements and into the skin. Disadvantages of such devices include the added complication and expense for adding a pressurizable liquid reservoir and complications due to the presence of a pressure-driven delivery system.

As disclosed in U.S. patent application Ser. No. 10/045,842, which is fully incorporated by reference herein, it is possible to have the drug that is to be delivered coated on the microprojections instead of contained in a physical reservoir. This eliminates the necessity of a separate physical reservoir and developing a drug formulation or composition specifically for the reservoir.

A drawback of the coated microprojection systems is that they are generally limited to delivery of a few hundred micrograms of the drug. A further drawback is that they are limited to a Bolus-type drug delivery profile.

It is therefore an object of the present invention to provide a transdermal drug delivery apparatus and method that substantially reduces or eliminates the aforementioned drawbacks and disadvantages associated with prior art drug delivery systems.

It is another object of the present invention to provide a transdermal drug delivery apparatus and method having an extended drug delivery profile.

It is another object of the present invention to provide a transdermal drug delivery apparatus and method that is capable of delivering up to 50 mg of drug per day.

It is another object of the present invention to provide a transdermal drug delivery apparatus having a hydrogel formulation and coated microprojection array that delivers drugs at an effective rate.

It is another object of the present invention to provide a transdermal drug delivery apparatus and method that enhances the delivery of a drug and, optionally, a vasoconstrictor through the stratum corneum of a patient via a plurality of coated stratum corneum-piercing microprojections.

### SUMMARY OF THE INVENTION

In accordance with the above objects and those that will be mentioned and will become apparent below, the apparatus for transdermally delivering a biologically active agent in accordance with this invention comprises (i) a gel pack containing a hydrogel formulation; and (ii) a microprojection member having top and bottom surfaces, a plurality of openings that extend through the microprojection member and a plurality of stratum corneum-piercing microprotrusions that project from the bottom surface of the microprojection member, the microprojection member being adapted to receive the gel pack whereby the hydrogel formulation flows through the microprojection member openings. Preferably, the hydrogel formulation comprises a water-based hydrogel.

In one embodiment of the invention, the hydrogel formulation comprises a polymeric material and, optionally, a surfactant. In one aspect of the invention, the polymeric material comprises a cellulose derivative. In a further aspect of the invention, the polymeric material is selected from the group consisting of hydroxyethylcellulose (HEC), hydroxypropylmethylcellulose (HPMC), hydroxypropycellulose (HPC), methylcellulose (MC), hydroxyethylmethylcellulose (HEMC), ethylhydroxyethylcellulose (EHEC), carboxymethyl cellulose (CMC), poly(vinyl alcohol), poly(ethylene oxide), poly(2-hydroxyethylmethacrylate), poly(n-vinyl pyrolidone), pluronics, and mixtures thereof. In a further aspect of the invention, the surfactant is selected from the group consisting of Tween 20 and Tween 80.

In the noted embodiment, the hydrogel formulation preferably includes at least one biologically active agent, which is preferably selected from the group consisting of leutinizing hormone releasing hormone (LHRH), LHRH analogs (such as goserelin, leuprolide, buserelin, triptorelin, gonadorelin, and napfarelin, menotropins (urofollitropin (FSH) and LH)), vasopressin, desmopressin, corticotrophin (ACTH), ACTH analogs such as ACTH (1-24), calcitonin, vasopressin, deamino[Val4, D-Arg8] arginine vasopressin, interferon alpha, interferon beta, interferon gamma, erythropoietin (EPO), granulocyte macrophage colony stimulating factor (GM-CSF), granulocyte colony stimulating factor (G-CSF), interleukin-10 (IL-10), glucagon, growth hormone releasing factor (GHRF), insulin, insulinotropin, calcitonin, octreotide, endorphin, TRN, NT-36 (chemical name: N[[(s)-4-oxo-2-azetidinyl]carbonyl]-L-histidyl-L-prolinamide), liprecin, aANF, bMSH, somatostatin, bradykinin, somatotropin, platelet-derived growth factor releasing factor, chymopapain, cholecystokinin, chorionic gonadotropin, epoprostenol (platelet aggregation inhibitor), glucagon, hirulog, interferons, interleukins, menotropins (urofollitropin (FSH) and LH), oxytocin, streptokinase, tissue plasminogen activator, urokinase, ANP, ANP clearance inhibitors, BNP, VEGF, angiotensin II antagonists, antidiuretic hormone agonists, bradykinin antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, parathyroid hormone antagonists, prostaglandin antagonists, pentigetide, protein C, protein S, renin inhibitors, thymosin alpha-1, thrombolytics, TNF, vasopressin antagonists analogs, alpha-I antitrypsin (recombinant), TGF-beta, fondaparinux, ardeparin, dalteparin, defibrotide, enoxaparin, hirudin, nadroparin, reviparin, tinzaparin, pentosan polysulfate, oligonucleotides and oligonucleotide derivatives such as formivirsen, alendronic acid, clodronic acid, etidronic acid, ibandronic acid, incadronic acid, pamidronic acid, risedronic acid, tiludronic acid, zoledronic acid, argatroban, RWJ 445167, RWJ-671818, fentanyl, remifentanyl, sufentanyl, alfentanyl, lofentanyl, carfentanyl, and mixtures thereof.

In a further embodiment of the invention, the hydrogel formulation includes at least one pathway patency modulator.

In yet another embodiment, the microprojection member includes a dialysis membrane that is disposed proximate the top surface of the microprojection member.

In accordance with a further embodiment of the invention, the apparatus for transdermally delivering a biologically active agent comprises (i) a gel pack containing a hydrogel formulation; (ii) a microprojection member having top and bottom surfaces, a plurality of openings that extend through the microprojection member and a plurality of stratum corneum-piercing microprotrusions that project from the bottom surface of the microprojection member, the microprojection member being adapted to receive the gel pack whereby the hydrogel formulation flows through the microprojection member openings; and (iii) a coating disposed on the microprojection member, the coating including at least one biologically active agent.

In the noted embodiment, the hydrogel formulation similarly comprises a polymeric material and, optionally, a surfactant. The hydrogel formulation is however optionally devoid of a biologically active material.

In one embodiment of the invention, the biologically active agent contained in the coating comprises a vaccine selected from the group consisting of conventional vaccines, recombinant protein vaccines, DNA vaccines and therapeutic cancer vaccines.

In a further embodiment, the biologically active agent is selected from the group consisting of leutinizing hormone releasing hormone (LHRH), LHRH analogs (such as goserelin, leuprolide, buserelin, triptorelin, gonadorelin, and napfarelin, menotropins (urofollitropin (FSH) and LH)), vasopressin, desmopressin, corticotrophin (ACTH), ACTH analogs such as ACTH (1-24), calcitonin, vasopressin, deamino[Val4, D-Arg8] arginine vasopressin, interferon alpha, interferon beta, interferon gamma, erythropoietin (EPO), granulocyte macrophage colony stimulating factor (GM-CSF), granulocyte colony stimulating factor (G-CSF), interleukin-10 (IL-10), glucagon, growth hormone releasing factor (GHRF), insulin, insulinotropin, calcitonin, octreotide, endorphin, TRN, NT-36 (chemical name: N[[(s)-4-oxo-2-azetidinyl]carbonyl]-L-histidyl-L-prolinamide), liprecin, aANF, bMSH, somatostatin, bradykinin, somatotropin, platelet-derived growth factor releasing factor, chymopapain, cholecystokinin, chorionic gonadotropin, epoprostenol (platelet aggregation inhibitor), glucagon, hirulog, interferons, interleukins, menotropins (urofollitropin (FSH) and LH), oxytocin, streptokinase, tissue plasminogen activator, urokinase, ANP, ANP clearance inhibitors, BNP, VEGF, angiotensin II antagonists, antidiuretic hormone agonists, bradykinin antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, parathyroid hormone antagonists, prostaglandin antagonists, pentigetide, protein C, protein S, renin inhibitors, thymosin alpha-1, thrombolytics, TNF, vasopressin antagonists analogs, alpha-i antitrypsin (recombinant), TGF-beta, fondaparinux, ardeparin, dalteparin, defibrotide, enoxaparin, hirudin, nadroparin, reviparin, tinzaparin, pentosan polysulfate, oligonucleotides and oligonucleotide derivatives such as formivirsen, alendronic acid, clodronic acid, etidronic acid, ibandronic acid, incadronic acid, pamidronic acid, risedronic acid, tiludronic acid, zoledronic acid, argatroban, RWJ 445167, RWJ-671818, fentanyl, remifentanyl, sufentanyl, alfentanyl, lofentanyl, carfentanyl, and mixtures thereof.

In another embodiment of the invention, the coating includes a vasoconstrictor, which is preferably selected from the group consisting of amidephrine, cafaminol, cyclopentamine, deoxyepinephrine, epinephrine, felypressin, indanazoline, metizoline, midodrine, naphazoline, nordefrin, octodrine, orinpressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and mixtures thereof.

In a further embodiment, the hydrogel formulation includes at least one pathway patency modulator.

In yet another embodiment, the microprojection member includes a dialysis member that is disposed proximate the top surface of the microprojection member.

In accordance with yet another embodiment of the invention, the apparatus for transdermally delivering a biologically active agent comprises (i) a gel pack containing a hydrogel formulation; and (ii) a microprojection member having top and bottom surfaces, a plurality of openings that extend through the microprojection member and a plurality of stratum corneum-piercing microprotrusions that project from the bottom surface of the microprojection member, the microprojection member including a solid film having at least one biologically active agent.

In one embodiment, the solid film is disposed proximate the top surface of the microprojection member. In another embodiment, the solid film is disposed proximate the bottom surface of the microprojection member.

In a preferred embodiment, the hydrogel formulation similarly comprises a polymeric material and, optionally, a surfactant. The polymeric material can either comprise a cellulose derivative or a polymeric material selected from the group consisting of hydroxyethylcellulose (HEC), hydroxypropylmethylcellulose (HPMC), hydroxypropycellulose (HPC), methylcellulose (MC), hydroxyethylmethylcellulose (HEMC), ethylhydroxyethylcellulose (EHEC), carboxymethyl cellulose (CMC), poly(vinyl alcohol), poly(ethylene oxide), poly(2-hydroxyethylmethacrylate), poly(n-vinyl pyrolidone), pluronics, and mixtures thereof and the optional surfactant is selected from the group consisting of Tween 20 and Tween 80. The hydrogel formulation is however optionally devoid of a biologically active material.

The biologically active agent disposed in the solid film can similarly comprise a vaccine selected from the group consisting of conventional vaccines, recombinant protein vaccines, DNA vaccines and therapeutic cancer vaccines or an agent selected from the group consisting of leutinizing hormone releasing hormone (LHRH), LHRH analogs (such as goserelin, leuprolide, buserelin, triptorelin, gonadorelin, and napfarelin, menotropins (urofollitropin (FSH) and LH)), vasopressin, desmopressin, corticotrophin (ACTH), ACTH analogs such as ACTH (1-24), calcitonin, vasopressin, deamino[Val4, D-Arg8] arginine vasopressin, interferon alpha, interferon beta, interferon gamma, erythropoietin (EPO), granulocyte macrophage colony stimulating factor (GM-CSF), granulocyte colony stimulating factor (G-CSF), interleukin-10 (1L-10), glucagon, growth hormone releasing factor (GHRF), insulin, insulinotropin, calcitonin, octreotide, endorphin, TRN, NT-36 (chemical name: N[[(s)-4-oxo-2-azetidinyl]carbonyl]-L-histidyl-L-prolinamide), liprecin, aANF, bMSH, somatostatin, bradykinin, somatotropin, platelet-derived growth factor releasing factor, chymopapain, cholecystokinin, chorionic gonadotropin, epoprostenol (platelet aggregation inhibitor), glucagon, hirulog, interferons, interleukins, menotropins (urofollitropin (FSH) and LH), oxytocin, streptokinase, tissue plasminogen activator, urokinase, ANP, ANP clearance inhibitors, BNP, VEGF, angiotensin II antagonists, antidiuretic hormone agonists, bradykinin antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, parathyroid hormone antagonists, prostaglandin antagonists, pentigetide, protein C, protein S, renin inhibitors, thymosin alpha-1, thrombolytics, TNF, vasopressin antagonists analogs, alpha-i antitrypsin (recombinant), TGF-beta, fondaparinux, ardeparin, dalteparin, defibrotide, enoxaparin, hirudin, nadroparin, reviparin, tinzaparin, pentosan polysulfate, oligonucleotides and oligonucleotide derivatives such as formivirsen, alendronic acid, clodronic acid, etidronic acid, ibandronic acid, incadronic acid, pamidronic acid, risedronic acid, tiludronic acid, zoledronic acid, argatroban, RWJ 445167, RWJ-671818, fentanyl, remifentanyl, sufentanyl, alfentanyl, lofentanyl, carfentanyl, and mixtures thereof.

In a further embodiment of the invention, the solid film includes a vasoconstrictor, which is preferably selected from the group consisting of amidephrine, cafaminol, cyclopentamine, deoxyepinephrine, epinephrine, felypressin, indanazoline, metizoline, midodrine, naphazoline, nordefrin, octodrine, orinpressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and mixtures thereof.

The method for transdermally delivering a biologically active agent to a patient, in accordance with one embodiment of the invention, comprises the steps of (i) providing a drug delivery apparatus having a gel pack and microprojection member, the gel pack containing a hydrogel formulation, the microprojection member having top and bottom surfaces, a plurality of openings that extend through the microprojection member and a plurality of stratum corneum-piercing microprotrusions that project from the bottom surface of the microprojection member, the microprojection member being adapted to receive the gel pack whereby the hydrogel formulation flows through the microprojection member openings; (ii) applying the microprojection member to the patient's skin; and (iii) placing the gel pack on the microprojection member after application of the microprojection member to the patient.

In one embodiment of the invention, the hydrogel formulation comprises a polymeric material and, optionally, a surfactant. In one aspect of the invention, the polymeric material comprises a cellulose derivative. In a further aspect of the invention, the polymeric material is selected from the group consisting of hydroxyethylcellulose (HEC), hydroxypropylmethylcellulose (HPMC), hydroxypropycellulose (HPC), methylcellulose (MC), hydroxyethylmethylcellulose (HEMC), ethylhydroxyethylcellulose (EHEC), carboxymethyl cellulose (CMC), poly(vinyl alcohol), poly(ethylene oxide), poly(2-hydroxyethylmethacrylate), poly(n-vinyl pyrolidone), pluronics, and mixtures thereof and, optionally, a surfactant selected from the group consisting of Tween 20 and Tween 80.

In a further embodiment of the invention, the hydrogel formulation includes at least one biologically active agent, which is preferably selected from the group consisting of leutinizing hormone releasing hormone (LHRH), LHRH analogs (such as goserelin, leuprolide, buserelin, triptorelin, gonadorelin, and napfarelin, menotropins (urofollitropin (FSH) and LH)), vasopressin, desmopressin, corticotrophin (ACTH), ACTH analogs such as ACTH (1-24), calcitonin, vasopressin, deamino[Val4, D-Arg8] arginine vasopressin, interferon alpha, interferon beta, interferon gamma, erythropoietin (EPO), granulocyte macrophage colony stimulating factor (GM-CSF), granulocyte colony stimulating factor (G-CSF), interleukin-10 (IL-10), glucagon, growth hormone releasing factor (GHRF), insulin, insulinotropin, calcitonin, octreotide, endorphin, TRN, NT-36 (chemical name: N[[(s)-4-oxo-2-azetidinyl]carbonyl]-L-histidyl-L-prolinamide), liprecin, aANF, bMSH, somatostatin, bradykinin, somatotropin, platelet-derived growth factor releasing factor, chymopapain, cholecystokinin, chorionic gonadotropin, epoprostenol (platelet aggregation inhibitor), glucagon, hirulog, interferons, interleukins, menotropins (urofollitropin (FSH) and LH), oxytocin, streptokinase, tissue plasminogen activator, urokinase, ANP, ANP clearance inhibitors, BNP, VEGF, angiotensin II antagonists, antidiuretic hormone agonists, bradykinin antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, parathyroid hormone antagonists, prostaglandin antagonists, pentigetide, protein C, protein S, renin inhibitors, thymosin alpha-1, thrombolytics, TNF, vasopressin antagonists analogs, alpha-I antitrypsin (recombinant), TGF-beta, fondaparinux, ardeparin, dalteparin, defibrotide, enoxaparin, hirudin, nadroparin, reviparin, tinzaparin, pentosan polysulfate, oligonucleotides and oligonucleotide derivatives such as formivirsen, alendronic acid, clodronic acid, etidronic acid, ibandronic acid, incadronic acid, pamidronic acid, risedronic acid, tiludronic acid, zoledronic acid, argatroban, RWJ 445167, RWJ-671818, fentanyl, remifentanyl, sufentanyl, alfentanyl, lofentanyl, carfentanyl, and mixtures thereof.

In another embodiment, the hydrogel formulation includes at least one pathway patency modulator.

In yet another embodiment, the microprojection member includes a dialysis membrane that is disposed proximate the top surface of the microprojection member.

In accordance with a further embodiment of the invention, the method for transdermally delivering a biologically active agent to a patient comprises the steps of (i) providing a drug delivery apparatus having a gel pack and a microprojection member, the gel pack containing a hydrogel formulation, the microprojection member having top and bottom surfaces, a plurality of openings that extend through the microprojection member and a plurality of stratum corneum-piercing microprotrusions that project from the bottom surface of the microprojection member, the microprojection member being adapted to receive the gel pack whereby the hydrogel formulation flows through the microprojection member openings; and a coating disposed on the microprojection member, the coating including a biologically active agent; (ii) applying the microprojection member to the patient's skin; and (iii) placing the gel pack on the microprojection member after application of the microprojection member to the patient.

In the noted embodiment, the hydrogel formulation similarly comprises a polymeric material and, optionally, a surfactant. The hydrogel is, however, optionally devoid of a biologically active material.

In one embodiment of the invention, the biologically active agent contained in the coating comprises a vaccine selected from the group consisting of conventional vaccines, recombinant protein vaccines, DNA vaccines and therapeutic cancer vaccines.

In a further embodiment, the biologically active agent is selected from the group consisting of leutinizing hormone releasing hormone (LHRH), LHRH analogs (such as goserelin, leuprolide, buserelin, triptorelin, gonadorelin, and napfarelin, menotropins (urofollitropin (FSH) and LH)), vasopressin, desmopressin, corticotrophin (ACTH), ACTH analogs such as ACTH (1-24), calcitonin, vasopressin, deamino[Val4, D-Arg8] arginine vasopressin, interferon alpha, interferon beta, interferon gamma, erythropoietin (EPO), granulocyte macrophage colony stimulating factor (GM-CSF), granulocyte colony stimulating factor (G-CSF), interleukin-10 (IL-10), glucagon, growth hormone releasing factor (GHRF), insulin, insulinotropin, calcitonin, octreotide, endorphin, TRN, NT-36 (chemical name: N[[(s)-4-oxo-2-azetidinyl]carbonyl]-L-histidyl-L-prolinamide), liprecin, aANF, bMSH, somatostatin, bradykinin, somatotropin, platelet-derived growth factor releasing factor, chymopapain, cholecystokinin, chorionic gonadotropin, epoprostenol (platelet aggregation inhibitor), glucagon, hirulog, interferons, interleukins, menotropins (urofollitropin (FSH) and LH), oxytocin, streptokinase, tissue plasminogen activator, urokinase, ANP, ANP clearance inhibitors, BNP, VEGF, angiotensin II antagonists, antidiuretic hormone agonists, bradykinin antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, parathyroid hormone antagonists, prostaglandin antagonists, pentigetide, protein C, protein S, renin inhibitors, thymosin alpha-1, thrombolytics, TNF, vasopressin antagonists analogs, alpha-i antitrypsin (recombinant), TGF-beta, fondaparinux, ardeparin, dalteparin, defibrotide, enoxaparin, hirudin, nadroparin, reviparin, tinzaparin, pentosan polysulfate, oligonucleotides and oligonucleotide derivatives such as formivirsen, alendronic acid, clodronic acid, etidronic acid, ibandronic acid, incadronic acid, pamidronic acid, risedronic acid, tiludronic acid, zoledronic acid, argatroban, RWJ 445167, RWJ-671818, fentanyl, remifentanyl, sufentanyl, alfentanyl, lofentanyl, carfentanyl, and mixtures thereof.

In another embodiment of the invention, the coating includes a vasoconstrictor, which is preferably selected from the group consisting of amidephrine, cafaminol, cyclopentamine, deoxyepinephrine, epinephrine, felypressin, indanazoline, metizoline, midodrine, naphazoline, nordefrin, octodrine, orinpressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and mixtures thereof.

In a further embodiment, the hydrogel formulation includes at least one pathway patency modulator.

In yet another embodiment, the microprojection member includes a dialysis member that is disposed proximate the top surface of the microprojection member.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is an exploded perspective view of one embodiment of the drug delivery system, according to the invention;

FIG. 2 is an exploded perspective view of one embodiment of the microprojection member, according to the invention;

FIG. 3 is an exploded perspective view of one embodiment of the gel pack assembled with the microprojection member, according to the invention;

FIG. 4 is a perspective view of one embodiment of the assembled drug delivery system, according to the invention;

FIG. 5 is a partial perspective view of one embodiment of a microprojection array, according to the invention;

FIG. 6 is an exploded diagrammatic view of the embodiment of the drug delivery system shown in FIGS. 1 through 4, according to the invention;

FIGS. 7 through 9 are diagrammatic views of various embodiment of the microprojection member, illustrating the incorporation and placement of a dialysis membrane and active agent film, according to the invention;

FIG. 10 is a sectioned side plane view of a retainer ring having a microprojection member disposed therein, according to the invention;

FIG. 11 is a perspective view of the retainer ring shown in FIG. 10;

FIG. 12 is a further diagrammatic view of the drug delivery system shown in FIGS. 1 through 4, illustrating the placement of the gel pack on the applied microprojection member, according to the invention;

FIG. 13 is a bar chart showing the global staining of pathways created by a microprojection array following contact with various formulations, according to the invention;

FIG. 14 is a bar chart showing the percentage of pathways created by a microprojection array that represent increasing staining scores following contact with various formulations, according to the invention;

FIG. 15 is a bar chart showing the percentage of pathways created by a microprojection array that represent increasing staining scores following contact with various formulations, according to the invention;

FIG. 16 is a graph showing the contact angle of various formulations;

FIG. 17 is a graph showing the viscosity of various formulations at different shear rates;

FIG. 18 is a graph showing the time dependent flux of an oligonucleotide through the skin of a living hairless guinea pig employing one embodiment of drug delivery system of the present invention;

FIG. 19 is a graph showing the concentration dependent flux of an oligonucleotide through the skin of a living hairless guinea pig; and

FIG. 20 is a bar chart showing the time dependent flux of desmopressin through the skin of a living hairless guinea pig.

### DETAILED DESCRIPTION OF THE INVENTION

Before describing the present invention in detail, it is to be understood that this invention is not limited to particularly exemplified materials, methods or structures as such may, of course, vary. Thus, although a number of materials and methods similar or equivalent to those described herein can be used in the practice of the present invention, the preferred materials and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “an active agent” includes two or more such agents; reference to “a microprojection” includes two or more such microprojections and the like.

### Definitions

The term “transdermal”, as used herein, means the delivery of an agent into and/or through the skin for local or systemic therapy.

The term “transdermal flux”, as used herein, means the rate of transdermal delivery.

The term “co-delivering”, as used herein, means that a supplemental agent(s) is administered transdermally either before the agent is delivered, before and during transdermal flux of the agent, during transdermal flux of the agent, during and after transdermal flux of the agent, and/or after transdermal flux of the agent. Additionally, two or more biologically active agents may be formulated in the hydrogel formulation(s) or solid film disposed on the microprojections resulting in co-delivery of the biologically active agents.

The term “biologically active agent”, as used herein, refers to a composition of matter or mixture containing a drug which is pharmacologically effective when administered in a therapeutically effective amount. Examples of such active agents include, without limitation, leutinizing hormone releasing hormone (LHRH), LHRH analogs (such as goserelin, leuprolide, buserelin, triptorelin, gonadorelin, and napfarelin, menotropins (urofollitropin (FSH) and LH)), vasopressin, desmopressin, corticotrophin (ACTH), ACTH analogs such as ACTH (1-24), calcitonin, vasopressin, deamino[Val4, D-Arg8] arginine vasopressin, interferon alpha, interferon beta, interferon gamma, erythropoietin (EPO), granulocyte macrophage colony stimulating factor (GM-CSF), granulocyte colony stimulating factor (G-CSF), interleukin-10 (IL-10), glucagon, growth hormone releasing factor (GHRF), insulin, insulinotropin, calcitonin, octreotide, endorphin, TRN, NT-36 (chemical name: N[[(s)-4-oxo-2-azetidinyl]carbonyl]-L-histidyl-L-prolinamide), liprecin, aANF, bMSH, somatostatin, bradykinin, somatotropin, platelet-derived growth factor releasing factor, chymopapain, cholecystokinin, chorionic gonadotropin, epoprostenol (platelet aggregation inhibitor), glucagon, hirulog, interferons, interleukins, menotropins (urofollitropin (FSH) and LH), oxytocin, streptokinase, tissue plasminogen activator, urokinase, ANP, ANP clearance inhibitors, BNP, VEGF, angiotensin II antagonists, antidiuretic hormone agonists, bradykinin antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, parathyroid hormone antagonists, prostaglandin antagonists, pentigetide, protein C, protein S, renin inhibitors, thymosin alpha-1, thrombolytics, TNF, vasopressin antagonists analogs, alpha-i antitrypsin (recombinant), TGF-beta, fondaparinux, ardeparin, dalteparin, defibrotide, enoxaparin, hirudin, nadroparin, reviparin, tinzaparin, pentosan polysulfate, oligonucleotides and oligonucleotide derivatives such as formivirsen, alendronic acid, clodronic acid, etidronic acid, ibandronic acid, incadronic acid, pamidronic acid, risedronic acid, tiludronic acid, zoledronic acid, argatroban, RWJ 445167, RWJ-671818, fentanyl, remifentanyl, sufentanyl, alfentanyl, lofentanyl, carfentanyl, and mixtures thereof.

The noted biologically active agents can also be in various forms, such as free bases, acids, charged or uncharged molecules, components of molecular complexes or nonirritating, pharmacologically acceptable salts. Further, simple derivatives of the active agents (such as ethers, esters, amides, etc.), which are easily hydrolyzed at body pH, enzymes, etc., can be employed.

The term “biologically active agent”, as used herein, also refers to a composition of matter or mixture containing a “vaccine” or other immunologically active agent or an agent which is capable of triggering the production of an immunologically active agent, and which is directly or indirectly immunologically effective when administered in an immunologically effective amount.

The term “vaccine”, as used herein, refers to conventional and/or commercially available vaccines, including, but not limited to, flu vaccines, Lyme disease vaccine, rabies vaccine, measles vaccine, mumps vaccine, chicken pox vaccine, small pox vaccine, hepatitis vaccine, pertussis vaccine, and diphtheria vaccine, recombinant protein vaccines, DNA vaccines and therapeutic cancer vaccines. The term “vaccine” thus includes, without limitation, antigens in the form of proteins, polysaccharides, oligosaccharides, lipoproteins, weakened or killed viruses such as cytomegalovirus, hepatitis B virus, hepatitis C virus, human papillomavirus, rubella virus, and varicella zoster, weakened or killed bacteria such as bordetella pertussis, clostridium tetani, corynebacterium diphtheriae, group A streptococcus, legionella pneumophila, neisseria meningitides, pseudomonas aeruginosa, streptococcus pneumoniae, treponema pallidum, and vibrio cholerae and mixtures thereof.

It is to be understood that more than one biologically active agent can be incorporated into the hydrogel formulations and/or coatings of this invention, and that the use of the term “active agent” in no way excludes the use of two or more such active agents or drugs.

The term “biologically effective amount” or “biologically effective rate” shall be used when the biologically active agent is a pharmaceutically active agent and refers to the amount or rate of the pharmacologically active agent needed to effect the desired therapeutic, often beneficial, result. The amount of active agent employed in the hydrogel formulations and coatings of the invention will be that amount necessary to deliver a therapeutically effective amount of the active agent to achieve the desired therapeutic result. In practice, this will vary widely depending upon the particular pharmacologically active agent being delivered, the site of delivery, the severity of the condition being treated, the desired therapeutic effect and the dissolution and release kinetics for delivery of the agent from the coating into skin tissues.

The term “biologically effective amount” or “biologically effective rate” shall also be used when the biologically active agent is an immunologically active agent and refers to the amount or rate of the immunologically active agent needed to stimulate or initiate the desired immunologic, often beneficial result. The amount of the immunologically active agent employed in the hydrogel formulations and coatings of the invention will be that amount necessary to deliver an amount of the active agent needed to achieve the desired immunological result. In practice, this will vary widely depending upon the particular immunologically active agent being delivered, the site of delivery, and the dissolution and release kinetics for delivery of the active agent into skin tissues.

The term “vasoconstrictor”, as used herein, refers to a composition of matter or mixture that narrows the lumen of blood vessels and, hence, reduces peripheral blood flow. Examples of suitable vasoconstrictors include, without limitation, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and the mixtures thereof.

The terms “microprojections” and “microprotrusions”, as used herein, refer to piercing elements that are adapted to pierce or cut through the stratum corneum into the underlying epidermis layer, or epidermis and dermis layers, of the skin of a living animal, particularly a mammal and more particularly a human.

In one embodiment of the invention, the microprojections have a projection length less than 1000 microns. In a further embodiment, the microprojections have a projection length of less than 500 microns, more preferably, less than 250 microns. The microprojections typically have a width and thickness of about 5 to 50 microns. The microprojections may be formed in different shapes, such as needles, blades, pins, punches, and combinations thereof.

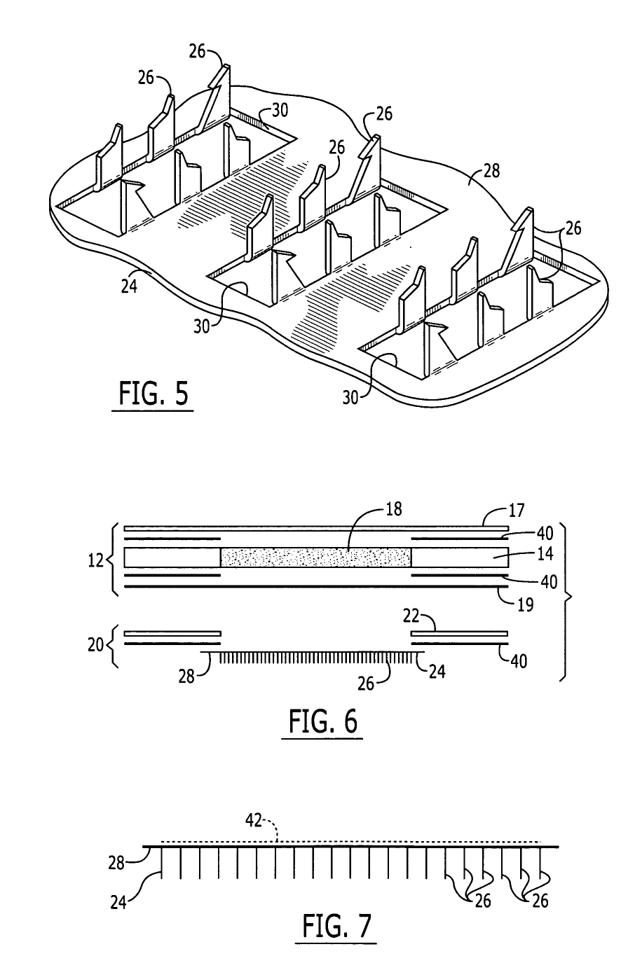

The term “microprojection array”, as used herein, refers to a plurality of microprojections arranged in an array for piercing the stratum corneum. The microprojection array may be formed by etching or punching a plurality of microprojections from a thin sheet and folding or bending the microprojections out of the plane of the sheet to form a configuration, such as that shown in FIG. 5. The microprojection array may also be formed in other known manners, such as by forming one or more strips having microprojections along an edge of each of the strip(s) as disclosed in U.S. Pat. No. 6,050,988.

References to the area of the sheet or member and reference to some property per area of the sheet or member are referring to the area bounded by the outer circumference or border of the sheet.

The term “solution” shall include not only compositions of fully dissolved components but also suspensions of components including, but not limited to, protein virus particles, inactive viruses, and split-virions.

The term “pattern coating”, as used herein, refers to coating an active agent onto selected areas of the microprojections. More than one active agent may be pattern coated onto a single microprojection array. Pattern coatings can be applied to the microprojections using known micro-fluid dispensing techniques such as micropipeting and ink jet coating.

As indicated above, the present invention comprises an apparatus and system for extended transdermal delivery of a biologically active agent (i.e., drug, active, etc.) to a patient. The system generally includes a gel patch that includes a hydrogel formulation and a microprojection member having a plurality of stratum corneum-piercing microprojections (or microprotrusions) extending therefrom.

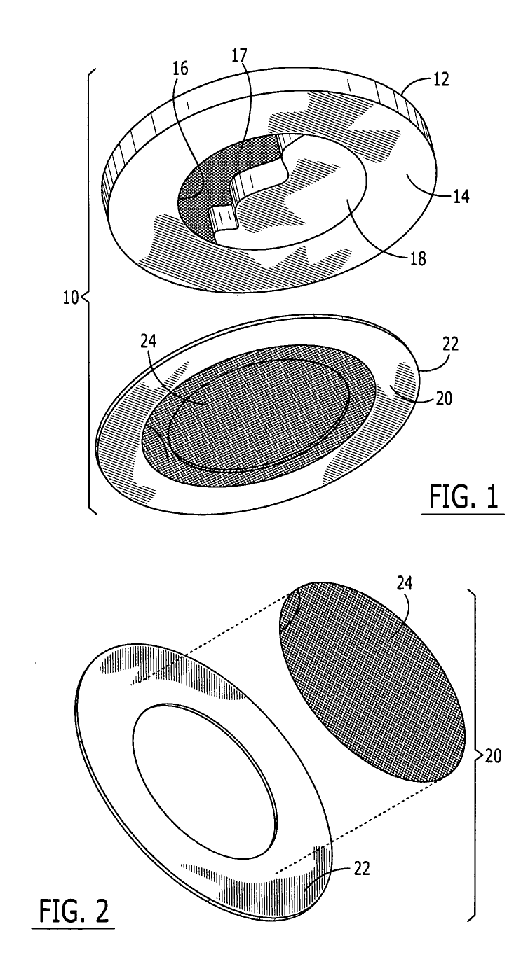

Referring now to FIG. 1, there is shown one embodiment of the drug delivery system 10 of the invention. As illustrated in FIG. 1, the system 10 includes a gel pack 12 and a microprojection member or patch 20.

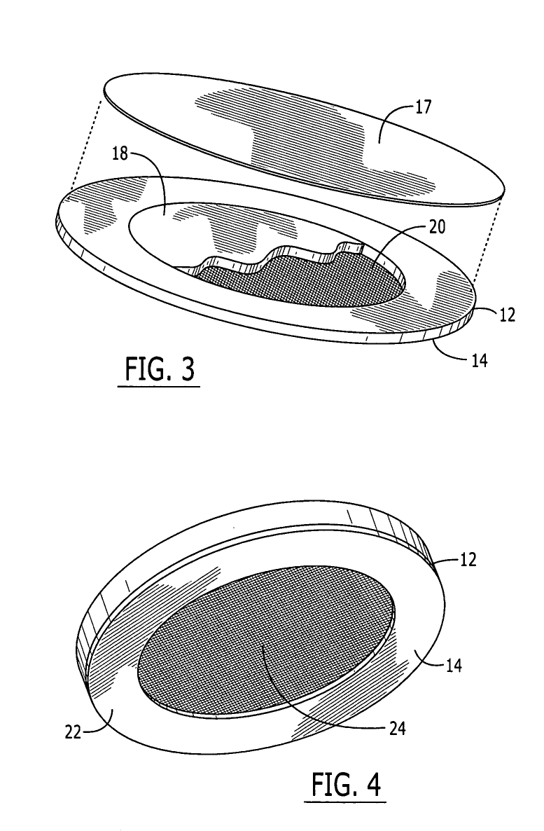

According to the invention, the gel pack 12 includes a housing or ring 14 having a centrally disposed reservoir or opening 16 that is adapted to receive a predetermined amount of a hydrogel formulation therein. The term “ring”, as used herein, is not limited to circular or oval shapes but also includes polygonal shapes, or polygonal shapes with rounded angles. As illustrated in FIGS. 1 and 3, the ring 14 further includes a backing member 17 that is disposed on the outer planar surface of the ring 14. Preferably, the backing member 17 is impermeable to the hydrogel formulation.

Preferably, the ring 14 is constructed out of a resilient polymeric material, such as PETG (polyethylene terephthalate, Glycol modified), polyethylene, or polyurethane. In a preferred embodiment, the ring 14 is constructed of closed or open-cell foam. The foam preferably, but not exclusively, comprises polyethylene, polyurethane, neoprene, natural rubber, SBR, butyl, butadiene, nitrile, EPDM, ECH, polystyrene, polyester, polyether, polypropylene, EVA, EMA, metallocene resin, PVC, and blends of the above.

Referring now to FIG. 2, the microprojection member 20 includes a backing membrane ring 22 and a microprojection array 24. Preferably, the backing membrane ring 22 is constructed out of a polymeric material, such as polyethylene, polyurethane and polypropylene. In a preferred embodiment, the backing membrane ring is constructed out of a polyethylene medical tape.

Referring now to FIG. 5, there is shown one embodiment of the microprojection array 24. As illustrated in FIG. 5, the microprojection array 24 includes a plurality of microprojections 26 that extend downward from one surface of a sheet or plate 28. The microprojections 26 are preferably sized and shaped to penetrate the stratum corneum of the epidermis when pressure is applied to the microprojection member 20.

The microprojections 26 are further adapted to form microslits in a body surface to increase the administration of a substance (e.g., hydrogel formulation) through the body surface. The term “body surface”, as used herein, refers generally to the skin of an animal or human.

The microprojections 26 are generally formed from a single piece of sheet material and are sufficiently sharp and long to puncture the stratum corneum of the skin. In the illustrated embodiment, the sheet 28 is formed with an opening 30 between the microprojections 26 to enhance the movement of the hydrogel formulation and, hence, active agent therethrough.

As discussed in detail below, the hydrogel formulations of the invention are released from the gel pack 12 through the openings 30, pass through microslits in the stratum corneum formed by the microprojections 26, migrate down the outer surfaces of the microprojections 26 and through the stratum corneum to achieve local or systemic therapy.

According to the invention, the number of microprojections 26 and openings 30 of the microprojection array 24 is variable with respect to the desired flux rate, agent being sampled or delivered, delivery or sampling device used (i.e., electrotransport, passive, osmotic, pressure-driven, etc.), and other factors that will be apparent to one of ordinary skill in the art. In general, the larger the number of microprojections per unit area (i.e., microprojection density), the more distributed the flux of the agent through the skin because there are more pathways.

In one embodiment of the invention, the microprojection density is at least approximately 10 microprojections/cm², more preferably, in the range of at least approximately 200-2000 microprojections/cm². In similar fashion, the number of openings per unit area through which the active agent passes is at least approximately 10 openings cm² and less than about 2000 openings/cm².

Further details of microprojection array 24 described above and other microprojection devices and arrays that can be employed within the scope of the invention are disclosed in U.S. Pat. Nos. 6,322,808, 6,230,051 B1 and Co-Pending U.S. application Ser. No. 10/045,842, which are incorporated by reference herein in their entirety.

Referring now to FIG. 6, the preferred construction of the gel pack 12 and microprojection member 20 will be described in detail. As illustrated in FIG. 6, the backing member 17 is adhered to the outer surface of the gel pack ring 14 via a conventional adhesive 40.

A strippable release liner 19 is similarly adhered to the outer surface of the gel pack ring 14 via a conventional adhesive 40. As described in detail below, the release liner 19 is removed prior to application of the gel pack 12 to the engaged microprojection member 20.

According to the invention, the backing membrane ring 22 is similarly adhered to the microprojection array 24 via a conventional adhesive. Optionally, the microprojection member 20 also includes a release liner (not shown) for maintaining the integrity of the member 20 when it is not in use. The release liner is similarly adapted to be stripped from the member 20 prior to applying the member 20 to the patient's skin.

In a further envisioned embodiment of the invention (not shown), an additional release liner is disposed on top of the backing membrane ring 22. According to the invention, this would substantially reduce or eliminate contamination of the piston of the applicator with skin/body fluids during application of the system.

In the noted envisioned embodiment, the top of the backing membrane ring 22 would be treated like the release side of a release liner, with an additional backing member, such as member 17, adhered to the top of the backing membrane ring 22 via a conventional adhesive. Following system application to skin, the entire assembly would be pealed off and the reservoir applied on the backing membrane ring 22.

Referring now to FIG. 7, in a further embodiment of the invention, the microprojection member 20 includes a dialysis (or rate controlling) membrane 42 that is disposed on at least the top surface of the microprojection array 24. According to the invention, if the hydrogel formulation 18 is devoid of a biologically active material, the membrane 42 preferably has a molecular weight (mw) cutoff that is less than the mw of the drug and is adapted to avoid diffusion of the drug in the hydrogel formulation. Conversely, if the hydrogel formulation 18 includes a biologically active agent, the membrane 42 preferably has a molecular weight (mw) cutoff that is more than the mw of the drug and is adapted to avoid diffusion of enzymes and/or bacteria in the hydrogel formulation.

As indicated above, in a preferred embodiment of the invention, the hydrogel formulation contains at least one biologically active agent. In an alternative embodiment of the invention, the hydrogel formulation is devoid of a biologically active agent and, hence, is merely a hydration mechanism.

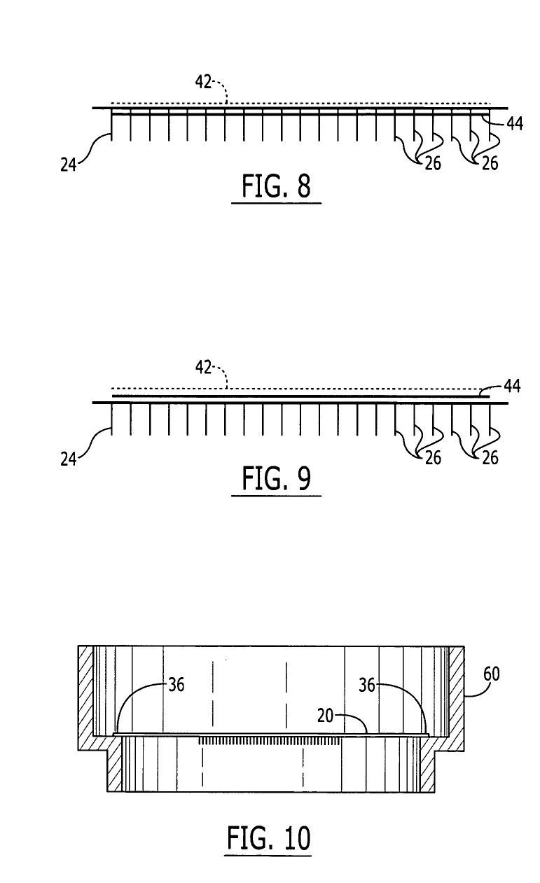

According to the invention, when the hydrogel formulation is devoid of a biologically active agent, the biologically active agent is either coated on the microprojection array 24, such as disclosed in U.S. application Ser. Nos. 10/045,842 and 10/674,626, which are incorporated by reference herein in their entirety, or contained in a solid film 44, such as disclosed in PCT Pub. No. WO 98/28037, which is similarly incorporated by reference herein in its entirety, on the skin side of the microprojection array 24 (see FIG. 8) or the top surface of the array 24 (see FIG. 9).

The solid film is typically made by casting a liquid formulation consisting of the biologically active agent, a polymeric material, such as hydroxyethylcellulose (HEC), hydroxypropylmethylcellulose (HPMC), hydroxypropycellulose (HPC), methylcellulose (MC), hydroxyethylmethylcellulose (HEMC), ethylhydroxyethylcellulose (EHEC), carboxymethyl cellulose (CMC), poly(vinyl alcohol), poly(ethylene oxide), poly(2 -hydroxyethylmethacrylate), poly(n-vinyl pyrolidone), or pluronics, a plasticising agent, such as glycerol, propylene glycol, or polyethylene glycol, a surfactant, such as tween 20 or tween 80, and a volatile solvent, such as water, isopropanol, or ethanol. Typically, this liquid formulation contains 1-20% biological agent, 5-40 wt. % polymer, 5-40 wt. % plasticiser, 0-2 wt. % surfactant, and the balance of volatile solvent. Following casting and subsequent evaporation of the solvent, a solid film is produced.

Preferably, the hydrogel formulations of the invention comprise water-based hydrogels. Hydrogels are preferred formulations because of their high water content and biocompatibility.

As is well known in the art, hydrogels are macromolecular polymeric networks that are swollen in water. Examples of suitable polymeric networks include, without limitation, hydroxyethylcellulose (HEC), hydroxypropylmethylcellulose (HPMC), hydroxypropycellulose (HPC), methylcellulose (MC), hydroxyethylmethylcellulose (HEMC), ethylhydroxyethylcellulose (EHEC), carboxymethyl cellulose (CMC), poly(vinyl alcohol), poly(ethylene oxide), poly(2-hydroxyethylmethacrylate), poly(n-vinyl pyrolidone), and pluronics. The most preferred polymeric materials are cellulose derivatives. These polymers can be obtained in various grades presenting different average molecular weight and therefore exhibit different rheological properties. Preferably, the concentration of the polymeric material is in the range of approximately 0.5-40 wt. % of the hydrogel formulation.

The hydrogel formulations of the invention preferably have sufficient surface activity to insure that the formulations exhibit adequate wetting characteristics, which are important for establishing optimum contact between the formulation and the microprojection array 24 and skin and, optionally, the solid film (e.g., film 44).

According to the invention, adequate wetting properties are achieved by incorporating a wetting agent in the hydrogel formulation. Optionally, a wetting agent can also be incorporated in the solid film.

As is well known in the art, wetting agents can generally be described as amphiphilic molecules. When a solution containing the wetting agent is applied to a hydrophobic substrate, the hydrophobic groups of the molecule bind to the hydrophobic substrate, while the hydrophilic portion of the molecule stays in contact with water. As a result, the hydrophobic surface of the substrate is not coated with hydrophobic groups of the wetting agent, making it susceptible to wetting by the solvent.

The noted wetting agents preferably include at least one surfactant. According to the invention, the surfactant(s) can be zwitterionic, amphoteric, cationic, anionic, or nonionic. Examples of surfactants include, sodium lauroamphoacetate, sodium dodecyl sulfate (SDS), cetylpyridinium chloride (CPC), dodecyltrimethyl ammonium chloride (TMAC), benzalkonium, chloride, polysorbates such as Tween 20 and Tween 80, other sorbitan derivatives such as sorbitan laurate, and alkoxylated alcohols such as laureth-4. Most preferred surfactants include Tween 20, Tween 80, and SDS.

Applicants have found that maximum wetting is observed at and above the critical micelle concentration (CMC). Wetting is also noticeable at concentrations as low as about one order of magnitude below the CMC.

Preferably, the wetting agents also include polymeric materials or polymers having amphiphilic properties. Examples of the noted polymers include, without limitation, cellulose derivatives, such as hydroxyethylcellulose (HEC), hydroxypropylmethylcellutose (HPMC), hydroxypropycellulose (HPC), methylcellulose (MC), hydroxyethylmethylcellulose (HEMC), or ethylhydroxyethylcellulose (EHEC), as well as pluronics.

Preferably, the concentration of the surfactant is in the range of approximately 0.001-2 wt. % of the hydrogel formulation. The concentration of the polymer that exhibits amphiphilic properties is preferably in the range of approximately 0.5-40 wt. % of the hydrogel formulation.

As will be appreciated by one having ordinary skill in the art, the noted wetting agents can be used separately or in combinations.

In a preferred embodiment, the hydrogel formulations of the invention contain at least one pathway patency modulator or “anti-healing agent”, such as those disclosed in Co-Pending U.S. application Ser. No. 09/950,436, which is incorporated by reference herein in its entirety. As set forth in the noted Co-Pending Application, the anti-healing agents prevent or diminish the skin's natural healing processes thereby preventing the closure of the pathways or microslits formed in the stratum corneum by the microprojection member 20. Examples of anti-healing agents include, without limitation, osmotic agents (e.g., sodium chloride), and zwitterionic compounds (e.g., amino acids).

The term “anti-healing agent”, as defined in the Co-Pending Application, further includes anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextran sulfate sodium, and EDTA.

According to the invention, the hydrogel formulations can also include a non-aqueous solvent, such as ethanol, propylene glycol, polyethylene glycol and the like, dyes, pigments, inert fillers, permeation enhancers, excipients, and other conventional components of pharmaceutical products or transdermal devices known in the art.

The hydrogel formulations of the invention exhibit adequate viscosity so that the formulation can be contained in the gel pack 12, keeps its integrity during the application process, and is fluid enough so that it can flow through the microprojection member openings 30 and into the skin pathways.

For hydrogel formulations that exhibit Newtonian properties, the viscosity of the hydrogel formulation is preferably in the range of approximately 2-30 Poises (P), as measured at 25° C. For shear-thinning hydrogel formulations, the viscosity, as measured at 25° C., is preferably in the range of 1.5-30 P or 0.5 and 10 P, at shear rates of 667/s and 2667/s, respectively. For dilatant formulations, the viscosity, as measured at 25° C., is preferably in the range of approximately 1.5-30 P, at a shear rate of 667/s.

As indicated, in a preferred embodiment of the invention, the hydrogel formulation contains at least one biologically active agent. Preferably, the biologically active agent comprises one of the aforementioned active agents, including, without limitation, leutinizing hormone releasing hormone (LHRH), LHRH analogs (such as goserelin, leuprolide, buserelin, triptorelin, gonadorelin, and napfarelin, menotropins (urofollitropin (FSH) and LH)), vasopressin, desmopressin, corticotrophin (ACTH), ACTH analogs such as ACTH (1-24), calcitonin, vasopressin, deamino[Val4, D-Arg8] arginine vasopressin, interferon alpha, interferon beta, interferon gamma, erythropoietin (EPO), granulocyte macrophage colony stimulating factor (GM-CSF), granulocyte colony stimulating factor (G-CSF), interleukin-10 (L-10), glucagon, growth hormone releasing factor (GHRF), insulin, insulinotropin, calcitonin, octreotide, endorphin, TRN, NT-36 (chemical name: N[[(s)-4-oxo-2-azetidinyl]carbonyl]-L-histidyl-L-prolinamide), liprecin, aANF, bMSH, somatostatin, bradykinin, somatotropin, platelet-derived growth factor releasing factor, chymopapain, cholecystokinin, chorionic gonadotropin, epoprostenol (platelet aggregation inhibitor), glucagon, hirulog, interferons, interleukins, menotropins (urofollitropin (FSH) and LH), oxytocin, streptokinase, tissue plasminogen activator, urokinase, ANP, ANP clearance inhibitors, BNP, VEGF, angiotensin II antagonists, antidiuretic hormone agonists, bradykinin antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, parathyroid hormone antagonists, prostaglandin antagonists, pentigetide, protein C, protein S, renin inhibitors, thymosin alpha-1, thrombolytics, TNF, vasopressin antagonists analogs, alpha-I antitrypsin (recombinant), TGF-beta, fondaparinux, ardeparin, dalteparin, defibrotide, enoxaparin, hirudin, nadroparin, reviparin, tinzaparin, pentosan polysulfate, oligonucleotides and oligonucleotide derivatives such as formivirsen, alendronic acid, clodronic acid, etidronic acid, ibandronic acid, incadronic acid, pamidronic acid, risedronic acid, tiludronic acid, zoledronic acid, argatroban, RWJ 445167, RWJ-671818, fentanyl, remifentanyl, sufentanyl, alfentanyl, lofentanyl, carfentanyl, and mixtures thereof.

As will be appreciated by one having ordinary skill in the art, the present invention has utility in connection with the delivery of biologically active agents or drugs within any of the broad class of drugs normally delivered though body surfaces and membranes, including skin. In general, this includes drugs in all of the major therapeutic areas.

According to the invention, when the hydrogel formulation contains one of the aforementioned active agents, the active agent can be present at a concentration in excess of saturation or below saturation. The amount of agent employed in the delivery device will be that amount necessary to deliver a therapeutically effective amount of the agent to achieve the desired result. In practice, this will vary widely depending upon the particular agent, the site of delivery, the severity of the condition, and the desired therapeutic effect. Thus, it is not practical to define a particular range for the therapeutically effective amount of agent incorporated into the method.

In one embodiment of the invention, the concentration of the active agent is in the range of at least 1-40 wt. % of the hydrogel formulation.

The biologically active agents can be in various forms, such as free bases, acids, charged or uncharged molecules, components of molecular complexes or nonirritating, pharmacologically acceptable salts. Also, simple derivatives of the agents (such as ethers, esters, amides, etc), which are easily hydrolyzed by body pH, enzymes, etc, can be employed. The agents can also be in solution, in suspension or a combination of both in the hydrogel formulation(s). Alternatively, the active agent can be a particulate.

As indicated, when the hydrogel formulation is devoid of a biologically active agent, the biologically active agent is either coated on the microprojection array 24 or contained in a solid film 44 on the skin side of the microprojection array 24 or the top surface of the array 24. According to the invention, the biologically active agent contained in the coating can also comprise any of the aforementioned biologically active agents and combinations thereof.

The hydrogel formulation and/or coating can further include at least one vasoconstrictor. Suitable vasoconstrictors include, without limitation, epinephrine, naphazoline, tetrahydrozoline indanazoline, metizoline, tramazoline, tymazoline, oxymetazoline, xylometazoline, amidephrine, cafaminol, cyclopentamine, deoxyepinephrine, epinephrine, felypressin, indanazoline, metizoline, midodrine, naphazoline, nordefrin, octodrine, omipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin and xylometazoline, and the mixtures thereof.

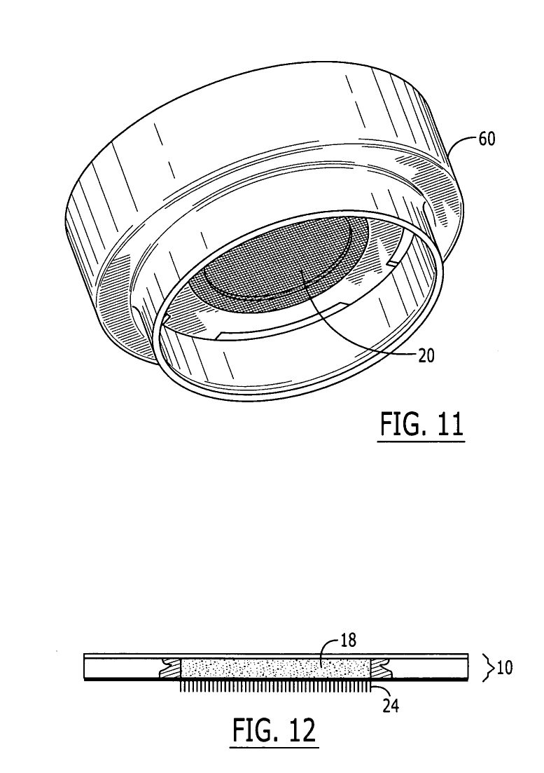

Referring now to FIGS. 10 and 11, for storage and application, the microprojection member 20 is preferably suspended in a retainer ring 60 by adhesive tabs 36, as described in detail in Co-Pending U.S. application Ser. No. 09/976,762 (Pub. No.2002/0091357), which is incorporated by reference herein in its entirety.

After placement of the microprojection member 20 in the retainer ring 60, the microprojection member 20 is applied to the patient's skin. Preferably, the microprojection member 20 is applied to the skin using an impact applicator, such as disclosed in Co-Pending U.S. application Ser. No. 09/976,798, which is incorporated by reference herein in its entirety.

After application of the microprojection member 20, the release liner 19 is removed from the gel pack 12. The gel pack 12 is then placed on the microprojection member 20 (see FIG. 12), whereby the hydrogel formulation 18 is released from the gel pack 12 through the openings 30 in the microprojection array 24, passes through the microslits in the stratum corneum formed by the microprojections 26, migrates down the outer surfaces of the microprojections 26 and through the stratum corneum to achieve local or systemic therapy.

It will be appreciated by one having ordinary skill in the art that in order to facilitate drug transport across the skin barrier, the present invention can also be employed in conjunction with a wide variety of iontophoresis or electrotransport systems, as the invention is not limited in any way in this regard. Illustrative electrotransport drug delivery systems are disclosed in U.S. Pat. Nos. 5,147,296, 5,080,646, 5,169,382 and 5,169383, the disclosures of which are incorporated by reference herein in their entirety.

The term “electrotransport” refers, in general, to the passage of a beneficial agent, e.g., a drug or drug precursor, through a body surface such as skin, mucous membranes, nails, and the like. The transport of the agent is induced or enhanced by the application of an electrical potential, which results in the application of electric current, which delivers or enhances delivery of the agent, or, for “reverse” electrotransport, samples or enhances sampling of the agent. The electrotransport of the agents into or out of the human body may by attained in various manners.

One widely used electrotransport process, iontophoresis, involves the electrically induced transport of charged ions. Electroosmosis, another type of electrotransport process involved in the transdermal transport of uncharged or neutrally charged molecules (e.g., transdermal sampling of glucose), involves the movement of a solvent with the agent through a membrane under the influence of an electric field. Electroporation, still another type of electrotransport, involves the passage of an agent through pores formed by applying an electrical pulse, a high voltage pulse, to a membrane.

In many instances, more than one of the noted processes may be occurring simultaneously to different extents. Accordingly, the term “electrotransport” is given herein its broadest possible interpretation, to include the electrically induced or enhanced transport of at least one charged or uncharged agent, or mixtures thereof, regardless of the specific mechanism(s) by which the agent is actually being transported. Additionally, other transport enhancing methods such as sonophoresis or piezoelectric devices can be used in conjunction with the invention.

When the invention is employed in conjunction with electrotransport, sonophoresis, or piezoelectric systems, the microprojection member 20 is first applied to the skin as explained above. The release liner 19 is removed from the gel pack 12, which is part of an electrotransport, sonophoresis, or piezoelectric system. This assembly is then placed on the microprojection member 20, whereby the hydrogel formulation 18 is released from the gel pack 12 through the openings 30 in the microprojection array 24, passes through the microslits in the stratum corneum formed by the microprojections 26, migrates down the outer surfaces of the microprojections 26 and through the stratum corneum to achieve local or systemic therapy with additional facilitation of drug transport provided by electrotransport, sonophoresis, or piezoelectric processes.

### EXAMPLES

The following examples are given to enable those skilled in the art to more clearly understand and practice the present invention. They should not be considered as limiting the scope of the invention but merely as being illustrated as representative thereof.

### Example 1

Hydrogel formulations having increasing concentrations of HEC (NATROSOL® 250 HHX PHARM, HERCULES Int. Lim. Netherlands, determined molecular weight: Mw 1890000, Mn 1050000), i.e., from 0% to 3%, and the surfactant Tween 80, at increasing concentrations varying from 0-0.25%, were prepared. In addition, methylene blue dye was present in the formulations at 1% for visualization of the skin pathways following hydrogel application. In order to be able to test low viscosity formulations, the system was slightly modified as explained below.

Application of the microprojection array was performed with an impact applicator in hairless rats. The system applied comprised a foam double adhesive ring (diameter 3.8 cm, thickness 0.16 cm) with a 2 cm² reservoir in the middle and a microprojection array having trapeziodally shaped microprojections bent at an angle of approximately 90° to the plane of the sheet, an area of 2 cm² and a microprojection density of 72 microprojections/cm². Each microprojection had a length of 500 microns.

Following microprojection application, 0.350 mL of the hydrogel formulation was dispensed into the gel pack reservoir and a backing membrane was applied to the adhesive outer surface of the ring to seal the system. After 1 min and 1 hour, the system was removed and the residual formulation washed from the skin. Excess dye was thoroughly removed with 70% isopropyl alcohol pads and a picture of the site was taken.

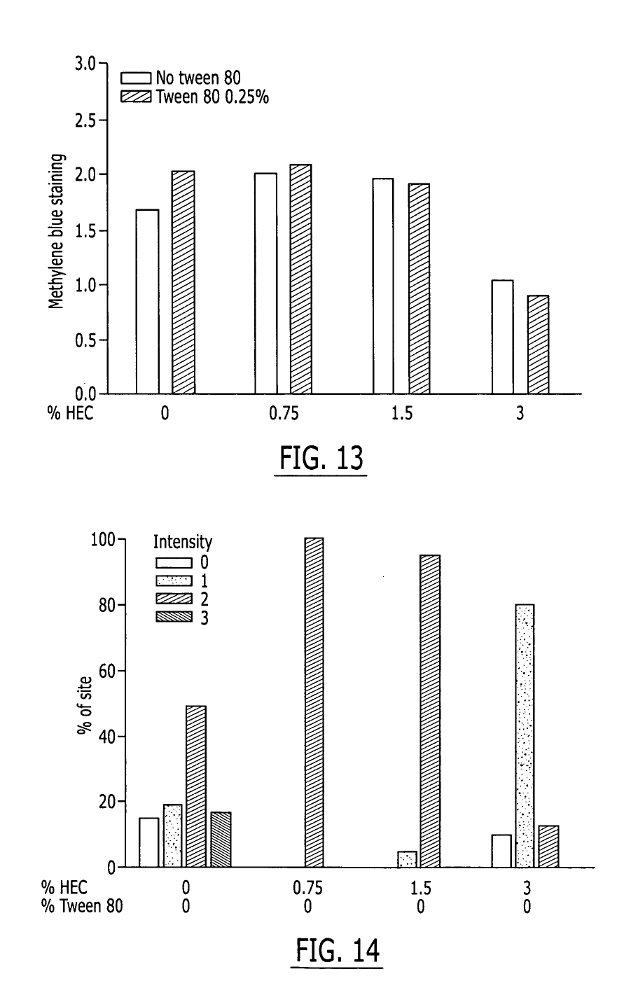

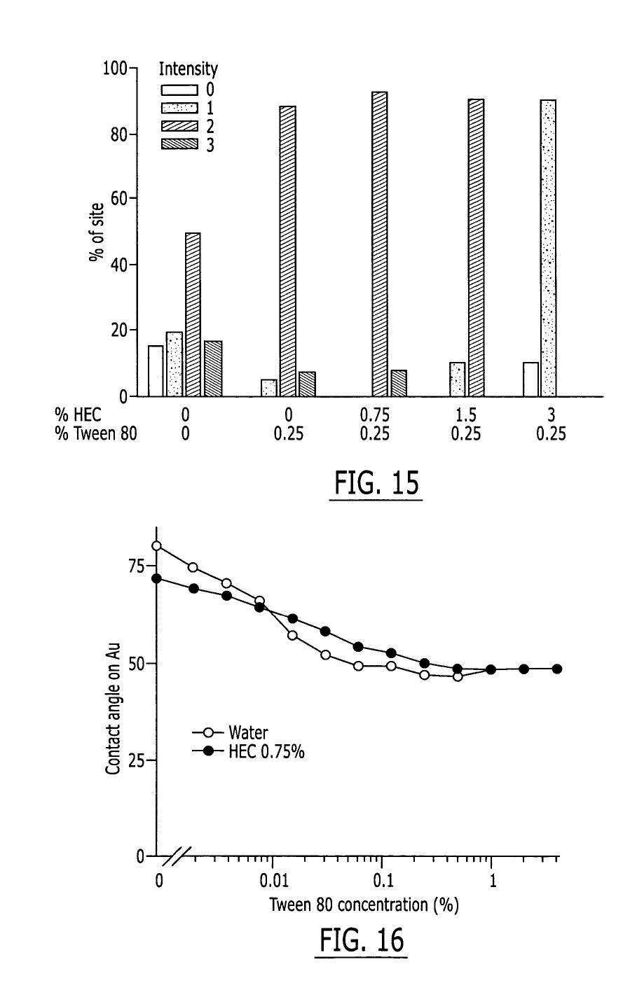

Dye staining of the pathways was evaluated visually by two people from the pictures on a 0 to 3 intensity scale corresponding to “no staining”, “faint”, “moderate”, and “intense staining”, respectively, and estimating the percentage of pathways that produced each score. From this data, average global staining was calculated (see FIG. 13) as well as the average percentages (see FIGS. 14 and 15).

Results at 1 min indicated that average global staining is only slightly improved by Tween 20 at 0.25% or low concentration of HEC and that high concentrations of HEC result in reduced staining (see FIG. 13). As reflected in FIGS. 14 and 15, heterogeneous staining was observed in the absence of the viscosity enhancing agent HEC or the surfactant Tween 80, indicating that poor contact of the formulation with the skin was achieved in the absence of these agents. Addition of HEC at 0.75% or Tween 80 at 0.25% improved staining homogeneity, indicating that these agents improve contact of the formulation with the skin.

Following 1 hour contact, all formulations showed maximal staining with good homogeneity (data not shown), indicating that good skin contact is achieved given additional time. In contrast, very highly viscous hydrogels prepared with 23% PVOH did not allow good skin contact even following prolonged wearing.

Additional experiments demonstrated that HEC at 1.5-3% offers optimal viscosity so that the hydrogel formulation can be contained in the gel patch, does not adhere to the release liner, and flows sufficiently to make contact with the microprojection array and the skin, resulting in homogeneous staining.

### Example 2

In order to understand the effective working range of surfactants and viscosity enhancing agents, the contact angle of formulations containing various concentrations of HEC and tween 80 were measured on a gold plate and the viscosity was measured at different shear rates. Results of contact angle measurements shown in FIG. 16 demonstrate that HEC 0.75% reduces the contact angle of water and that Tween 80 also decreases the contact angle at concentrations as low as 0.002%.

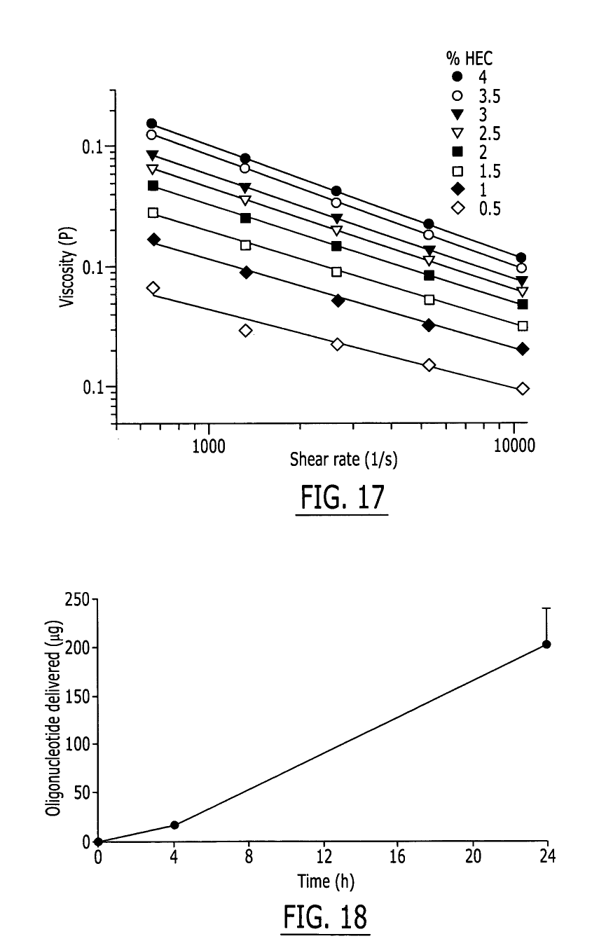

Evaluation of viscosity of HEC-containing formulations yielded the data shown in FIG. 17 demonstrating non-Newtownian, shear-thinning, behavior. For this type of hydrogel formulation, the optimal viscosity, as measured at 25° C., to achieve good skin contact is preferably in the range of 1.5-30 P or 0.5 and 10 P, at shear rates of 667/s and 2667/s, respectively, and most preferably in the range of 3-10 P or 1 and 3 P, at shear rates of 667/s and 2667/s, respectively. Addition of the surfactants Tween 20 or Tween 80 to these formulations did not affect viscosity (data not shown).

### Example 3

As is well known in the art, oligonucleotides are highly negatively charged compounds that typically do not penetrate the skin significantly without the use of penetration enhancers or physical disruption of the skin barrier. In this experiment, an oligonucleotide was delivered by passive diffusion through pathways in the skin of hairless guinea pigs (HGPs) created by a microprojection array.

The system included a foam double adhesive ring (diameter 3.8 cm, thickness 0.16 cm) with a drug containing hydrogel formulation having a skin contact area of 2 cm² in the middle, and a stainless steel microprojection array having a thickness of 0.025 mm, an area of 2 cm², trapezoidally shaped microprojections bent at an angle of approximately 90° to the plane of the sheet, and a microprojection density of 241 microprojections/cm². Each microprojection had a length of 500 microns.

The formulation comprised 0.35 mL of a hydrogel formulation containing tritiated oligonucleotide at various concentrations in 2% HEC.

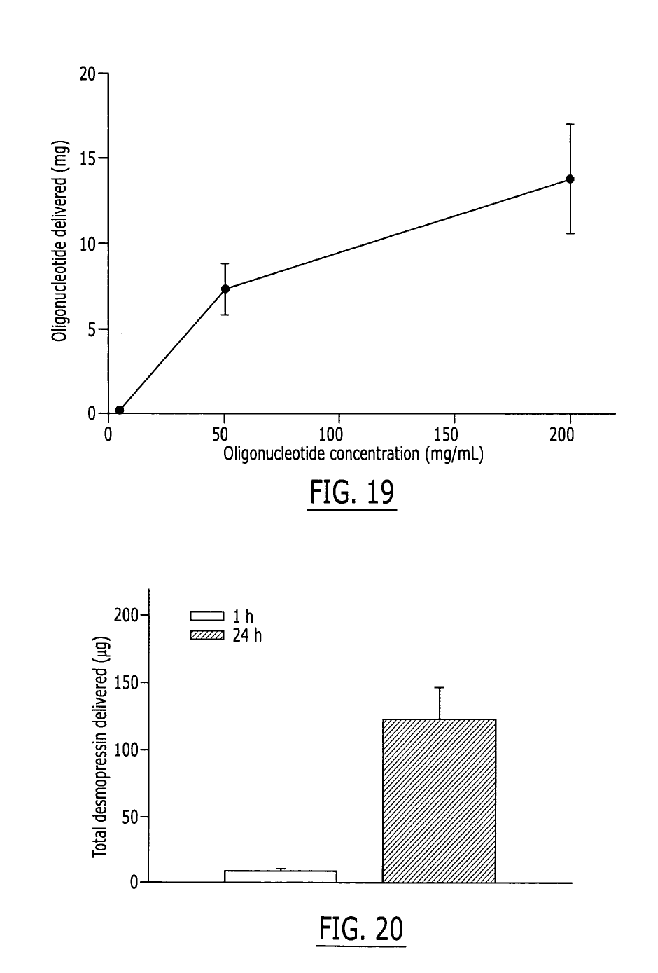

At various times after application, three (3) systems from each group were removed and the residual drug washed from the skin. The amount of drug penetrated during these time intervals was determined by measuring oligonucleotide liver content (previous studies had shown that following systemic administration in HGP's, about 50% of the oligonucleotide accumulates in the liver). The results reflected a time dependant (see FIG. 18) and concentration dependant (see FIG. 19) flux of the oligonucleotide through the skin.

### Example 4

An experiment was conducted to test the concept of the hydratable system using the peptide desmopressin. A system similar to that presented in Example 2 was provided. The microprojection array was constructed of titanium and had a microprojection density of about 300 microprojections/cm². Each microprojection had a length of 200 microns.

The system included a 2 cm² solid film containing 5 mg tritiated desmopressin. The thin film was prepared by casting a 20 mil thick aqueous solution comprised of 10 wt. % HPMC 2910 USP and 20 wt. % glycerol. The film was dried and punched into 2 cm² discs. Each disc was imbibed with a 20 wt. % ³H desmopressin solution and subsequently dried. The solid film was subsequently disposed proximate the top surface of the microprojection member. The gel pack or gel reservoir contained 0.120 mL of 2% HEC (NATROSOL® 250 HHX) in water.

Following application of the microprojection solid film system in HGPs, the gel pack was placed on top of the microprojection member, as illustrated in FIG. 12. At 1 h and 24 h after application, three (3) systems from each group of HGPs were removed and the residual drug washed form the skin. The amount of the drug penetrated during these times intervals was determined by measuring urinary excretion of tritium (previous studies had shown that in HGPs, 71% of ³H desmopressin injected intravenously is excreted in urine). The results indicated a time dependant flux of desmopressin though the skin (see FIG. 20).

From the foregoing description, one of ordinary skill in the art can easily ascertain that the present invention, among other things, provides an effective and efficient means for extending the transdermal delivery of biologically active agents to a patient.

As will be appreciated by one having ordinary skill in the art, the present invention provides many advantages, such as:
Transdermal delivery of up to 50 mg per day of biologically active agents with one application.
Extended delivery profiles of biologically active agents.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

1. An apparatus for transdermally delivering a biologically active agent, comprising:
a gel pack containing a hydrogel formulation; and
a microprojection member having top and bottom surfaces, a plurality of openings that extend through said microprojection member and a plurality of stratum corneum-piercing microprotrusions that project from said bottom surface of said microprojection member, said microprojection member being adapted to receive said gel pack whereby said hydrogel formulation flows through said microprojection member openings.

2. The apparatus of claim 1, wherein said hydrogel formulation comprises a water-based hydrogel.

3. The apparatus of claim 2, wherein said hydrogel formulation comprises a polymeric material.

4. The apparatus of claim 3, wherein said polymeric material comprises a cellulose derivative.

5. The apparatus of claim 3, wherein said polymeric material is selected from the group consisting of EHEC, CMC, poly(vinyl alcohol), poly(ethylene oxide), poly(2-hydroxyethylmethacrylate), poly(n-vinyl pyrtolidone) and mixtures thereof.

6. The apparatus of claim 1, wherein said hydrogel formulation includes at least one biologically active agent.

7. The apparatus of claim 6, wherein said biologically active agent is selected from the group consisting of a leutinizing hormone releasing hormone (LHRH), LHRH analogs, vasopressin, desmopressin, corticotropin (ACTH), ACTH analogs, including ACTH (1-24), calcitonin, parathyroid hormone (PTH), vasopressin, deamino [Val4, D-Arg8] arginine vasopressin, interferon alpha, interferon beta, interferon gamma, erythropoietin (EPO), granulocyte macrophage colony stimulating factor (GM-CSF), granulocyte colony stimulating factor (G-CSF), interleukin-10 (IL-10), glucagon, growth hormone releasing hormone (GHRH), growth hormone releasing factor (GHRF), insulin, insultropin, calcitonin, octreotide, endorphin, TRN, N[[(s)-4-oxo-2-azetidinyl]carbonyl]-L-histidyl-L-prolinamide, liprecin, pituitary hormones, including HGH, HMG and desmopressin acetate, follicle luteoids, aANF, growth factors, including growth factor releasing factor (GFRF), bMSH, GH, somatostatin, bradykinin, somatotropin, platelet-derived growth factor releasing factor, asparaginase, bleomycin sulfate, chymopapain, cholecystokinin, chorionic gonadotropin, corcticotropin (ACTH), erythropoietin, epoprostenol (platelet aggregation inhibitor), gluagon, HCG, hirulog, hyaluronidase, interferon, interleukins, menotropins (urofollitropin (FSH) and LH), oxytocin, streptokinase, tissue plasminogen activator, urokinase, vasopressin, desmopressin, ANP, ANP clearance inhibitors, BNP, VEGF, angiotensin II antagonists, antidiuretic hormone agonists, bradykinn antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, parathyroid hormone antagonists, prostaglandin antagonists, pentigetide, protein C, protein S, renin inhibitors, thymosin alpha-1, thrombolytics, TNF, vasopressin antagonists analogs, alpha-I antitrypsin (recombinant), TGF-beta, and mixtures thereof.

8. The apparatus of claim 1, wherein said hydrogel formulation includes at least one pathway patency modulator.

9. The apparatus of claim 1, wherein said hydrogel formulation has a viscosity in the range of approximately 2-10 poises, said viscosity being measured at 25° C.

10. The apparatus of claim 1, wherein said microprojection member includes a dialysis membrane, said dialysis membrane being disposed proximate said top surface of said microprojection member.

11. The apparatus of claim 1, wherein said delivery system includes a retainer ring that is adapted to cooperate with a patch applicator.

12. The apparatus of claim 11, wherein said retainer includes a microprojection member seat adapted to receive said microprojection member.

13. The apparatus of claim 12, wherein said backing membrane of the microprojection member comprises a ring.

14. The apparatus of claim 13, wherein said backing membrane ring includes adhesive tabs adapted to adhere to said microprojection patch seat.

15. The apparatus of claim 13, wherein, following application of the microprojection member to the skin, said backing membrane ring is used as a template for subsequent application of a gel pack.

16. An apparatus for transdermally delivering a biologically active agent, comprising:
a gel pack containing a hydrogel formulation;
a microprojection member having top and bottom surfaces, a plurality of openings that extend through said microprojection member and a plurality of stratum corneum-piercing microprotrusions that project from said bottom surface of said microprojection member, said microprojection member being adapted to receive said gel pack whereby said hydrogel formulation flows through said microprojection member openings; and
a coating disposed on said microprojection member, said coating including a biologically active agent.

17. The apparatus of claim 16, wherein said hydrogel formulation comprises polymeric material.

18. The apparatus of claim 17, wherein said polymeric material comprises a cellulose derivative.

19. The apparatus of claim 17, wherein said polymeric material is selected from the group consisting of EHEC, CMC, poly(vinyl alcohol), poly(ethylene oxide), poly(2-hydroxyethylmethacrylate), poly(n-vinyl pyrtoidone) and mixtures thereof.

20. The apparatus of claim 16, wherein said biologically active agent comprises a vaccine selected from the group consisting of conventional vaccines, recombinant protein vaccines, DNA vaccines and therapeutic cancer vaccines.

21. The apparatus of claim 16, wherein said biologically active agent is selected from the group consisting of a leutinizing hormone releasing hormone (LHRH), LHRH analogs, vasopressin, desmopressin, corticotropin (ACTH), ACTH analogs, including ACTH (1-24), calcitonin, parathyroid hormone (PTH), vasopressin, deamino [Val4, D-Arg8] arginine vasopressin, interferon alpha, interferon beta, interferon gamma, erythropoietin (EPO), granulocyte macrophage colony stimulating factor (GM-CSF), granulocyte colony stimulating factor (G-CSF), interleukin-10 (1L-10), glucagon, growth hormone releasing hormone (GHRH), growth hormone releasing factor (GHRF), insulin, insultropin, calcitonin, octreotide, endorphin, TRN, N[[(s)-4-oxo-2-azetidinyl]carbonyl]-L-histidyl-L-prolinamide, liprecin, pituitary hormones, including HGH, HMG and desmopressin acetate, follicle luteoids, aANF, growth factors, including growth factor releasing factor (GFRF), bMSH, GH, somatostatin, bradykinin, somatotropin, platelet-derived growth factor releasing factor, asparaginase, bleomycin sulfate, chymopapain, cholecystokinin, chorionic gonadotropin, corcticotropin (ACTH), erythropoietin, epoprostenol (platelet aggregation inhibitor), gluagon, HCG, hirulog, hyaluronidase, interferon, interleukins, menotropins (urofollitropin (FSH) and LH), oxytocin, streptokinase, tissue plasminogen activator, urokinase, vasopressin, desmopressin, ANP, ANP clearance inhibitors, BNP, VEGF, angiotensin II antagonists, antidiuretic hormone agonists, bradykinn antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, parathyroid hormone antagonists, prostaglandin antagonists, pentigetide, protein C, protein S, renin inhibitors, thymosin alpha-1, thrombolytics, TNF, vasopressin antagonists analogs, alpha-I antitrypsin (recombinant), TGF-beta, and mixtures thereof.

22. The apparatus of claim 16, wherein said coating includes a vasoconstrictor.

23. The apparatus of claim 22, wherein said vasoconstrictor is selected from the group consisting of amidephrine, cafaminol, cyclopentamine, deoxyepinephrine, epinephrine, felypressin, indanazoline, metizoline, midodrine, naphazoline, nordefrin, octodrine, orinpressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and mixtures thereof.

24. The apparatus of claim 23, wherein said vasoconstrictor comprises in the range of 0.1-10.0 wt. % of said coating.

25. The apparatus of claim 16, wherein said coating comprises a dry coating, said dry coating comprising an aqueous solution prior to drying.

26. The apparatus of claim 16, wherein said coating thickness is less than 10 microns.

27. The apparatus of claim 16, wherein each of said plurality of stratum corneum-piercing microprotrusions has a length less than approximately 1000 microns.

28. The apparatus of claim 27, wherein each of said plurality of stratum corneum-piercing microprotrusions has a length less than approximately 500 microns.

29. The apparatus of claim 27, wherein each of said plurality of stratum corneum-piercing microprotrusions has a thickness in the range of approximately 5-50 microns.

30. The apparatus of claim 16, wherein said coating has a thickness less than 50 microns.

31. The apparatus of claim 30, wherein said coating thickness is less than 10 microns.

32. The apparatus of claim 16, wherein each of said plurality of stratum corneum-piercing microprotrusions includes in the range of 1 microgram to 1 milligram of said biologically active agent.

33. The apparatus of claim 16, wherein said hydrogel formulation includes at least one pathway patency modulator.

34. The apparatus of claim 16, wherein said microprojection member includes a dialysis member, said dialysis membrane being disposed proximate said top surface of said microprojection member.

35. An apparatus for transdermally delivering a biologically active agent, comprising:
a gel pack containing a hydrogel formulation; and
a microprojection member having top and bottom surfaces, a plurality of openings that extend through said microprojection member and a plurality of stratum corneum-piercing microprotrusions that project from said bottom surface of said microprojection member, said microprojection member including a dry film having a biologically active agent.

36. The apparatus of claim 35, wherein said dry film is disposed proximate said top surface of said microprojection member.

37. The apparatus of claim 35, wherein said dry film is disposed proximate said bottom surface of said microprojection member.

38. The apparatus of claim 35, wherein said hydrogel formulation comprises polymeric material.

39. The apparatus of claim 38, wherein said polymeric material comprises a cellulose derivative.

40. The apparatus of claim 38, wherein said polymeric material is selected from the group consisting of EHEC, CMC, poly(vinyl alcohol), poly(ethylene oxide), poly(2-hydroxyethylmethacrylate), poly(n-vinyl pyrtoidone) and mixtures thereof.

41. The apparatus of claim 35, wherein said biologically active agent comprises a vaccine selected from the group consisting of conventional vaccines, recombinant protein vaccines, DNA vaccines and therapeutic cancer vaccines.

42. The apparatus of claim 35, wherein said biologically active agent is selected from the group consisting of a leutinizing hormone releasing hormone (LHRH), LHRH analogs, vasopressin, desmopressin, corticotropin (ACTH), ACTH analogs, including ACTH (1-24), calcitonin, parathyroid hormone (PTH), vasopressin, deamino [Val4, D-Arg8] arginine vasopressin, interferon alpha, interferon beta, interferon gamma, erythropoietin (EPO), granulocyte macrophage colony stimulating factor (GM-CSF), granulocyte colony stimulating factor (G-CSF), interleukin-10 (IL-10), glucagon, growth hormone releasing hormone (GHRH), growth hormone releasing factor (GHRF), insulin, insultropin, calcitonin, octreotide, endorphin, TRN, N[[(s)-4-oxo-2-azetidinyl]carbonyl]-L-histidyl-L-prolinamide, liprecin, pituitary hormones, including HGH, HMG and desmopressin acetate, follicle luteoids, aANF, growth factors, including growth factor releasing factor (GFRF), bMSH, GH, somatostatin, bradykinin, somatotropin, platelet-derived growth factor releasing factor, asparaginase, bleomycin sulfate, chymopapain, cholecystokinin, chorionic gonadotropin, corcticotropin (ACTH), erythropoietin, epoprostenol (platelet aggregation inhibitor), gluagon, HCG, hirulog, hyaluronidase, interferon, interleukins, menotropins (urofollitropin (FSH) and LH), oxytocin, streptokinase, tissue plasminogen activator, urokinase, vasopressin, desmopressin, ANP, ANP clearance inhibitors, BNP, VEGF, angiotensin II antagonists, antidiuretic hormone agonists, bradykinn antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, parathyroid hormone antagonists, prostaglandin antagonists, pentigetide, protein C, protein S, renin inhibitors, thymosin alpha-1, thrombolytics, TNF, vasopressin antagonists analogs, alpha-I antitrypsin (recombinant), TGF-beta, and mixtures thereof.

43. The apparatus of claim 35, wherein said dry film includes a vasoconstrictor.

44. The apparatus of claim 43, wherein said vasoconstrictor is selected from the group consisting of amidephrine, cafaminol, cyclopentamine, deoxyepinephrine, epinephrine, felypressin, indanazoline, metizoline, midodrine, naphazoline, nordefrin, octodrine, orinpressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and mixtures thereof.

45. A method of transdermally delivering a biologically active agent to a patient, the method comprising the steps of:
providing a drug delivery apparatus having a gel pack and microprojection member, said gel pack containing a hydrogel formulation, said microprojection member having top and bottom surfaces, a plurality of openings that extend through said microprojection member and a plurality of stratum corneum-piercing microprotrusions that project from said bottom surface of said microprojection member, said microprojection member being adapted to receive said gel pack whereby said hydrogel formulation flows through said microprojection member openings;
applying said microprojection member to the patient's skin; and
placing said gel pack on said microprojection member after said application of said microprojection member to the patient.

46. The method of claim 45, wherein said hydrogel formulation comprises a water-based hydrogel.

47. The method of claim 46, wherein said hydrogel formulation comprises a polymeric material.

48. The method of claim 47, wherein said polymeric material comprises a cellulose derivative.

49. The method of claim 47, wherein said polymeric material is selected from the group consisting of EHEC, CMC, poly(vinyl alcohol), poly(ethylene oxide), poly(2-hydroxyethylmethacrylate), poly(n-vinyl pyrtolidone) and mixtures thereof.

50. The method of claim 47, wherein said hydrogel formulation includes at least one biologically active agent.

51. The method of claim 47, wherein said biologically active agent is selected from the group consisting of a leutinizing hormone releasing hormone (LHRH), LHRH analogs, vasopressin, desmopressin, corticotropin (ACTH), ACTH analogs, including ACTH (1-24), calcitonin, parathyroid hormone (PTH), vasopressin, deamino [Val4, D-Arg8] arginine vasopressin, interferon alpha, interferon beta, interferon gamma, erythropoietin (EPO), granulocyte macrophage colony stimulating factor (GM-CSF), granulocyte colony stimulating factor (G-CSF), interleukin-10 (L-10), glucagon, growth hormone releasing hormone (GHRH), growth hormone releasing factor (GHRF), insulin, insultropin, calcitonin, octreotide, endorphin, TRN, N[[(s)-4-oxo-2-azetidinyl]carbonyl]-L-histidyl-L-prolinamide, liprecin, pituitary hormones, including HGH, HMG and desmopressin acetate, follicle luteoids, aANF, growth factors, including growth factor releasing factor (GFRF), bMSH, GH, somatostatin, bradykinin, somatotropin, platelet-derived growth factor releasing factor, asparaginase, bleomycin sulfate, chymopapain, cholecystokinin, chorionic gonadotropin, corcticotropin (ACTH), erythropoietin, epoprostenol (platelet aggregation inhibitor), gluagon, HCG, hirulog, hyaluronidase, interferon, interleukins, menotropins (urofollitropin (FSH) and LH), oxytocin, streptokinase, tissue plasminogen activator, urokinase, vasopressin, desmopressin, ANP, ANP clearance inhibitors, BNP, VEGF, angiotensin II antagonists, antidiuretic hormone agonists, bradykinn antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, parathyroid hormone antagonists, prostaglandin antagonists, pentigetide, protein C, protein S, renin inhibitors, thymosin alpha-1, thrombolytics, TNF, vasopressin antagonists analogs, alpha-I antitrypsin (recombinant), TGF-beta, and mixtures thereof.

52. The method of claim 47, wherein said hydrogel formulation includes at least one pathway potency modulator.

53. The method of claim 47, wherein said hydrogel formulation has a viscosity in the range of approximately 2-10 poises, said viscosity being at 25° C.

54. The method of claim 47, wherein said microprojection member includes a dialysis membrane, said dialysis membrane being disposed proximate said top surface of said microprojection member.

55. A method of transdermally delivering a biologically active agent to a patient, comprising the steps of:
providing a drug delivery apparatus having a gel pack and a microprojection member, said gel pack containing a hydrogel formulation, said microprojection member having top and bottom surfaces, a plurality of openings that extend through said microprojection member and a plurality of stratum corneum-piercing microprotrusions that project from said bottom surface of said microprojection member, said microprojection member being adapted to receive said gel pack whereby said hydrogel formulation flows through said microprojection member openings; and a coating disposed on said microprojection member, said coating including a biologically active agent;
applying said microprojection member to the patient's skin; and
placing said gel pack on said microprojection member after said application of said microprojection member to the patient.

56. The method of claim 55, wherein said hydrogel formulation comprises polymeric material.

57. The method of claim 56, wherein said polymeric material comprises a cellulose derivative.

58. The method of claim 56, wherein said polymeric material is selected from the group consisting of EHEC, CMC, poly(vinyl alcohol), poly(ethylene oxide), poly(2-hydroxyethylmethacrylate), poly(n-vinyl pyrtoidone) and mixtures thereof.

59. The method of claim 55, wherein said biologically active agent comprises a vaccine selected from the group consisting of conventional vaccines, recombinant protein vaccines, DNA vaccines and therapeutic cancer vaccines.

60. The apparatus of claim 55, wherein said biologically active agent is selected from the group consisting of a leutinizing hormone releasing hormone (LHRH), LHRH analogs, vasopressin, desmopressin, corticotropin (ACTH), ACTH analogs, including ACTH (1-24), calcitonin, parathyroid hormone (PTH), vasopressin, deamino [Val4, D-Arg8] arginine vasopressin, interferon alpha, interferon beta, interferon gamma, erythropoietin (EPO), granulocyte macrophage colony stimulating factor (GM-CSF), granulocyte colony stimulating factor (G-CSF), interleukin-10 (IL-10), glucagon, growth hormone releasing hormone (GHRH), growth hormone releasing factor (GHRF), insulin, insultropin, calcitonin, octreotide, endorphin, TRN, N[[(s)-4-oxo-2-azetidinyl]carbonyl]-L-histidyl-L-prolinamide, liprecin, pituitary hormones, including HGH, HMG and desmopressin acetate, follicle luteoids, aANF, growth factors, including growth factor releasing factor (GFRF), bMSH, GH, somatostatin, bradykinin, somatotropin, platelet-derived growth factor releasing factor, asparaginase, bleomycin sulfate, chymopapain, cholecystokinin, chorionic gonadotropin, corcticotropin (ACTH), erythropoietin, epoprostenol (platelet aggregation inhibitor), gluagon, HCG, hirulog, hyaluronidase, interferon, interleukins, menotropins (urofollitropin (FSH) and LH), oxytocin, streptokinase, tissue plasminogen activator, urokinase, vasopressin, desmopressin, ANP, ANP clearance inhibitors, BNP, VEGF, angiotensin II antagonists, antidiuretic hormone agonists, bradykinn antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, parathyroid hormone antagonists, prostaglandin antagonists, pentigetide, protein C, protein S, renin inhibitors, thymosin alpha-1, thrombolytics, TNF, vasopressin antagonists analogs, alpha-i antitrypsin (recombinant), TGF-beta, and mixtures thereof.

61. The method of claim 55, wherein said coating includes a vasoconstrictor selected from the group consisting of amidephrine, cafaminol, cyclopentamine, deoxyepinephrine, epinephrine, felypressin, indanazoline, metizoline, midodrine, naphazoline, nordefrin, octodrine, orinpressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and mixtures thereof.

62. The method of claim 55, wherein said hydrogel formulation includes at least one pathway patency modulator.

63. The apparatus of claim 55, wherein said microprojection member includes a dialysis member, said dialysis membrane being disposed proximate said top surface of said microprojection member.
